(19) United States
(12) Patent Application Publication
(10) Pub. No.: US 2015/0119911 A1
(43) Pub. Date: Apr. 30, 2015

(54) TONGUE SCRAPER

(71) Applicant: Gladys MCKENZIE, Dundas (AU)

(72) Inventor: Gladys MCKENZIE, Dundas (AU)

(21) Appl. No.: 14/522,551

(22) Filed: Oct. 23, 2014

(30) Foreign Application Priority Data
Oct. 25, 2013 (AU) 2013904137

(51) Int. Cl. A61B 17/24

(52) U.S. Cl. CPC A61B 17/244; A61B 2017/00862
USPC 606/161

(57) ABSTRACT

The tongue cleaner includes an elongated generally straight scraping member terminated at each end by grip structures. The scraping member is constructed of a flexible and resilient material allowing the scraping member to be bendable from its generally straight configuration into at least a curved shape, ‘C’ shape, ‘V’ shape or ‘U’ shape configuration. The scraping member resiliently returns to its straight configuration in the absence of any bending forces. The flexible and resilient material is preferably a plastic, silicone, rubber, metal, wood or a combination of such materials. The preferred material is silicone rubber.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention relates to a device for cleaning a tongue. Specifically, the present invention relates to a tongue cleaner or tongue scraper.

2. Description of Related Art Including Information Disclosed Under 37 CFR 1.97 and 37 CFR 1.98.

The cleaning of a person's tongue assists with the maintenance of oral hygiene, reducing the build-up of bacteria, food debris, fungi, dead cells, and other unwanted residue on the tongue. One method of cleaning the tongue involves the use of a tongue scraper for scraping off the unwanted residue from the tongue.

Conventional tongue scrapers typically take the form of a longitudinal handle having attached to an end thereof one or more blades which extend perpendicular to the handle. The blades are similar in shape to a squeegee for cleaning windows. The blades are placed on the tongue and run across the tongue through movement of the longitudinal member. The blade removes the unwanted residue as it is run along the tongue. Other conventional tongue scrapers comprise a curved metal strip that is inserted into the mouth. A thin edge of the metal strip is run along the tongue to remove the unwanted residue.

Conventional tongue scrapers have not seen wide adoption despite the oral hygiene benefits of tongue cleaning. The poor adoption of tongue scrapers may be due to a perceived repulsion for the act, and/or a dislike of conventional tongue scrapers which are perceived to look like dental equipment.

The present invention seeks to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect, the present invention provides a tongue cleaner comprising a scraping member having at least one scraping edge, the scraping member comprising a resilient flexible material allowing the scraping member to be bendable from a generally straight configuration into a curved or bent configuration.

In a preferred embodiment, the scraping member defines two or more scraping edges.

In a preferred embodiment, a first scraping edge has a different scraping characteristic to that of a second scraping edge.

In a preferred embodiment, the scraping member is adapted to bend into a ‘C’ shape, ‘V’ shape or ‘U’ shape configuration.

In a preferred embodiment, the scraping member defines three scraping edges.

In a preferred embodiment, the scraping member has a substantially planar portion.

In a preferred embodiment, the scraping member has a generally T-shaped cross-section.

In a preferred embodiment, the scraping member terminates at each end in a grip structure, the grip structure facilitating gripping and manipulation of the tongue cleaner.

In a preferred embodiment, the at least one grip structure defines an aperture passing therethrough, the aperture adapted to receive therein a supporting element on which to hang the tongue cleaner.

In a preferred embodiment, each grip structure has a shape comprising one or more of: spherical, hemispherical, cubed, polygon, circular, heart shape, a knob handle, a diamond handle, a square handle, a rectangle handle or an oval handle.

In a preferred embodiment, the scraping member is constructed from a plastic, silicone, rubber, metal, wood or any combination thereof.

In a preferred embodiment, a length of the scraping member is between 100 mm and 200 mm.

In a preferred embodiment, width of each grip structure is between 10 mm and 20 mm.

In a preferred embodiment, a diameter of each grip structure is between 15 mm and 20 mm.

In a preferred embodiment, the different scraping characteristic comprises one or more of a different angle, shape, texture, hardness and/or contour.

In a preferred embodiment, a material of the scraping member is treated with an anti-bacterial agent.

In another preferred embodiment, the scraping member has a generally band-shaped cross-section.

In another preferred embodiment, the scraping member has a generally round-shaped cross-section.

Other aspects of the invention are also disclosed.

### BRIEF DESCRIPTION OF THE DRAWINGS

Notwithstanding any other forms which may fall within the scope of the present invention, preferred embodiments of the present invention will now be described, by way of examples only, with reference to the accompanying drawings.

FIG. 1 is a perspective view of a tongue cleaner in accordance with a first preferred embodiment of the present invention.

FIG. 1A is a cross sectional view of the tongue cleaner along line A-A of FIG. 1.

FIG. 2 illustrates the tongue cleaner from (a) top plan, (b) side elevation, and (c) end view.

FIG. 3 (a) and (b) illustrates a schematic view of use of the tongue cleaner.

FIG. 4 shows a tongue cleaner according to a second preferred embodiment of the present invention, where (a) is a perspective view, (b) is a top view, (c) is a front view and (d) is a partial perspective cross-section view.

FIG. 5 shows perspective views of a plurality of the tongue cleaners of FIG. 4 in different colours.

FIG. 6 shows a tongue cleaner according to a third preferred embodiment of the present invention, where (a) is a perspective view, (b) is a top view, (c) is a front view and (d) is a partial perspective cross-section view.

### DETAILED DESCRIPTION OF THE DRAWINGS

It should be noted in the following description that like or the same reference numerals in different embodiments denote the same or similar features.

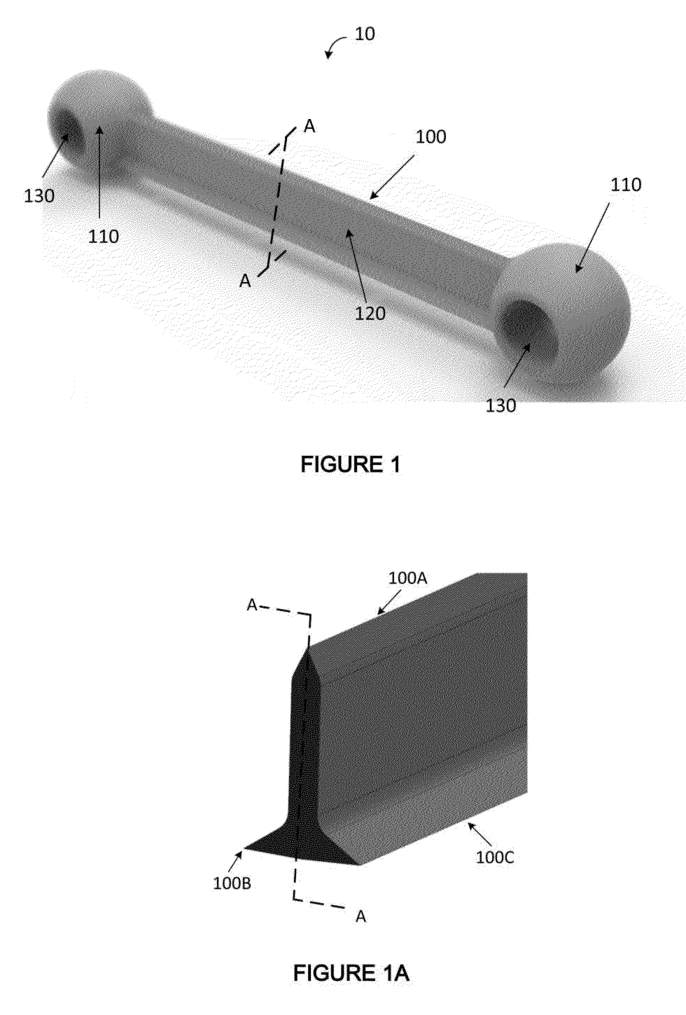

FIG. 1 illustrates a tongue cleaner 10 according to a preferred embodiment of the present invention. The tongue cleaner 10 includes an elongated generally straight scraping member 100 terminated at each end by grip structures 110.

The scraping member 100 is constructed of a flexible and resilient material allowing the scraping member 100 to be bendable from its generally straight configuration into at least a curved shape, ‘C’ shape, ‘V’ shape or ‘U’ shape configuration. The scraping member 100 resiliently returns to its straight configuration in the absence of any bending forces. The flexible and resilient material is preferably a plastic, silicone, rubber, metal, wood or a combination of such materials. The preferred material is silicone rubber.

Referring to FIG. 1A, a cross section of the scraping member 100 taken along line A-A in FIG. 1 is shown. The scraping member 100 according to a preferred embodiment comprises a generally T-shaped cross-section and defines three scraping edges 100A, 1008, and 100C.

In the orientation shown, the cross-section is an upside down T-shape, with the first scraping edge 100A provided along a top edge of the scraping member 100. Second and third scraping edges 1008, 100C are defined as lower edges protruding in opposing directions perpendicularly from a plane of the scraping member.

The scraping edges 100A, 1008, 100C provide differently angled edges in use against the tongue surface to achieve different scraping effects. Alternatively, or in addition to providing different angles in use, the scraping edges 100A, 1008, 100C may also have different textures, different edge profiles (e.g. jagged, sawtooth, etc.), different hardness, different shapes and contours, and the like. Multiple scraping edges of the same characteristic may also be provided to increase the usable life of the tongue cleaner 10. It is to be understood that the scraping member 100 may have only two scraping edges, or may have three or more scraping edges.

Referring back to FIG. 1, the scraping member 100 is preferably shaped to define a thin section 120. The thin section 120 assists with the bending of the tongue cleaner 10 in use.

The grip structures 110 provide a structure to facilitate the gripping and manipulation of the tongue cleaner 10. The grip structures 110 have a nodular shape to assist with grip, and preferably define an aperture 130 therethrough. The aperture 130 is sized and shape to receive therein a supporting element such as a hook or other protrusion, to facilitate storage of the tongue cleaner 10 by, for example, hanging the tongue cleaner on a bathroom mirror or wall from the supporting element. The grip structures 110 may have a spherical, hemispherical, cubed, polygon, or other three dimensional shape. Other possible shapes of the grip structure includes a planar shape such as a flat round circular shape or a flat heart shape, a knob style handle, a diamond style handle, a square handle, a rectangle handle or an oval handle. The grip structure 110 adds to the appearance appeal of the tongue cleaner 10.

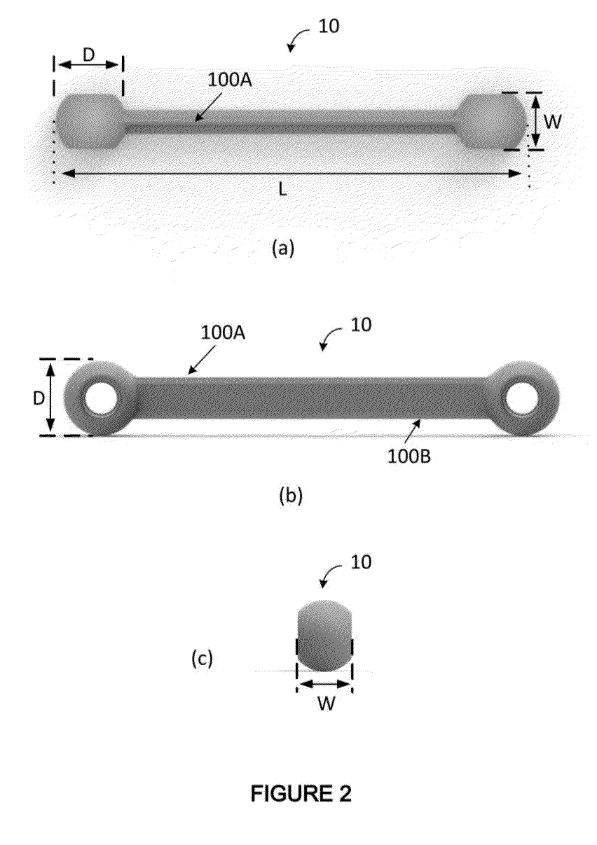

Referring to FIG. 2, the tongue cleaner 10 is illustrated from various views. As illustrated, the grip structures 110 have a width W of between 10 mm and 20 mm, and preferably around 14 mm, and a diameter D of between 15 mm and 20 mm, and preferably around 18 mm. The tongue cleaner 10 has a length L of between 100 mm and 150 mm, and preferably around 118 mm. The tongue cleaner 10 is provided in a number of different sizes to accommodate mouth and tongue sizes of children, teenagers, adults, and elderly people.

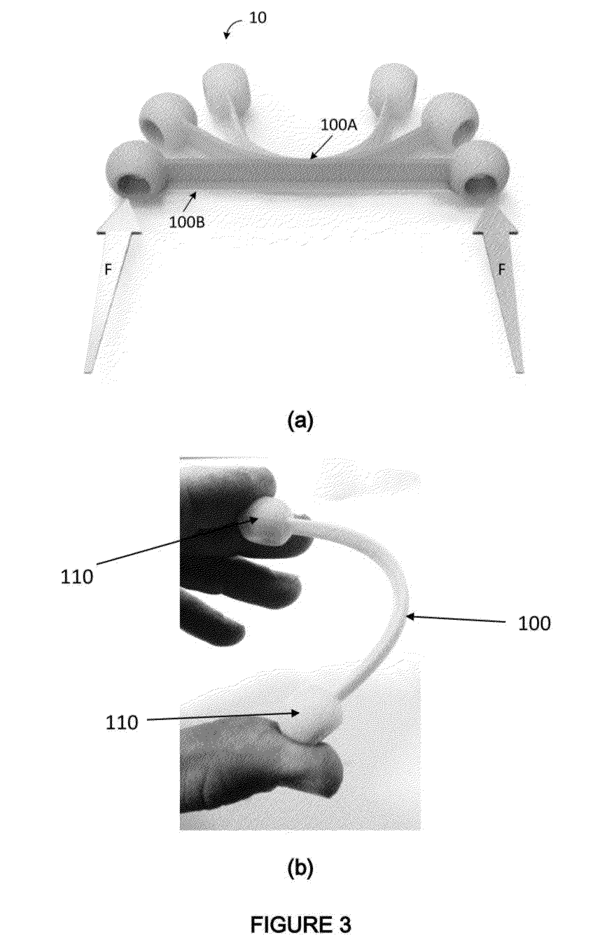

Referring to FIG. 3, use of the tongue cleaner 10 is illustrated. The tongue cleaner 10 is adapted to be gripped by the fingers of one hand of a user, with the thumb engaging one grip structure 110 and a forefinger or middle finger engaging the other grip structure 110. Alternatively, both hands can be used where each hand grips a respective grip structure 110.

The user's fingers apply a bending force having components F at each end of the tongue scraper 10 via the grip structures 110, typically by forcing the grip structures 110 towards each other. The received force bends the tongue cleaner 10 into an approximate ‘C’ shape or ‘U’ shape.

The bent tongue cleaner 10 is inserted into the mouth and placed in contact with the tongue such that one or more of the scraping edges 100A, 100B or 100C is pressed against the tongue. The tongue cleaner 10 is then slowly pulled out of the mouth with the scraping edge 100A, 100B or 100C remaining in contact with the tongue such that any unwanted residue such as of bacteria, food debris, fungi, dead cells, and other unwanted residue are scraped from the tongue. This process is repeated as desired.

To employ a different scraping edge 100A, 100B or 100C, the tongue cleaner 10 is rotated until the desired scraping edge 100A, 100B or 100C faces the tongue, then bent to be inserted in and slowly pulled out of the mouth as described above.

The tongue scraper 10 has a generally straight shape and configuration in the absence of any bending forces, and resiliently returns to this configuration. In combination with the attractive shape and colour, this provides the tongue scraper 10 with a very attractive appearance compared to conventional tongue scrapers leading to an increase in the use of the tongue scraper 10.

The material of the tongue cleaner 10 is preferably treated with anti-bacterial agents to keep the device free of germs. The material is further colour treated to provide the tongue cleaner 10 with bright and attractive colours. The colours, together with the apertures 130 for facilitating hanging of the tongue cleaner 10 in a prominent place of the bathroom, increase the chances that children in particular will use the tongue cleaner 10 and include tongue cleaning as part of their daily oral hygiene habit.

Wear and tear of the tongue scraping device 10 through use will cause the scraping edges 100A, 1008, 100C to dull, and also cause cavities and recesses to form over the surfaces of the tongue scraping device 10. Dull scraping edges 100A, 1008, 100C reduce the effectiveness of the scraping action, and cavities and recesses on the surface encourage the growth of bacteria and trapping of unwanted debris. Accordingly, the tongue cleaner 10 is constructed as a limited use, disposable item.

The tongue scraping device 10 of the present invention is constructed as a simple one piece, integrated device of a readily available and cost effective material. Assembly is not required, facilitating mass and rapid fabrication of the tongue scraping device 10 by methods such as moulding, 3D printing, stamping, and casting.

The tongue scraping device 10 is preferably made available in bulk to consumers, to promulgate the image of the tongue scraping device being a limited use, highly disposable item. In this manner, the repulsion of having perceptually unhygienic tongue scrapers present around the living environment is ameliorated. Similarly, the perceived repulsion of tongue scraping is also ameliorated as the disposable nature of the tongue cleaner 10, its bright colours, and anti-bacterially treated material promulgate a sense of tongue cleaning being quick, non-messy, and chemically protected. In another embodiment, a bulk pack of the tongue cleaners 10 can be provided with each tongue cleaner being a one use disposable item.

Whilst preferred embodiments of the present invention have been described, it will be apparent to skilled persons that modifications can be made to the embodiments described.

The tongue scraper 10 for example can have other shaped cross-sections such as L-shaped, X-shaped, H-shaped, I-shaped, J-shaped, K-shaped or similar.

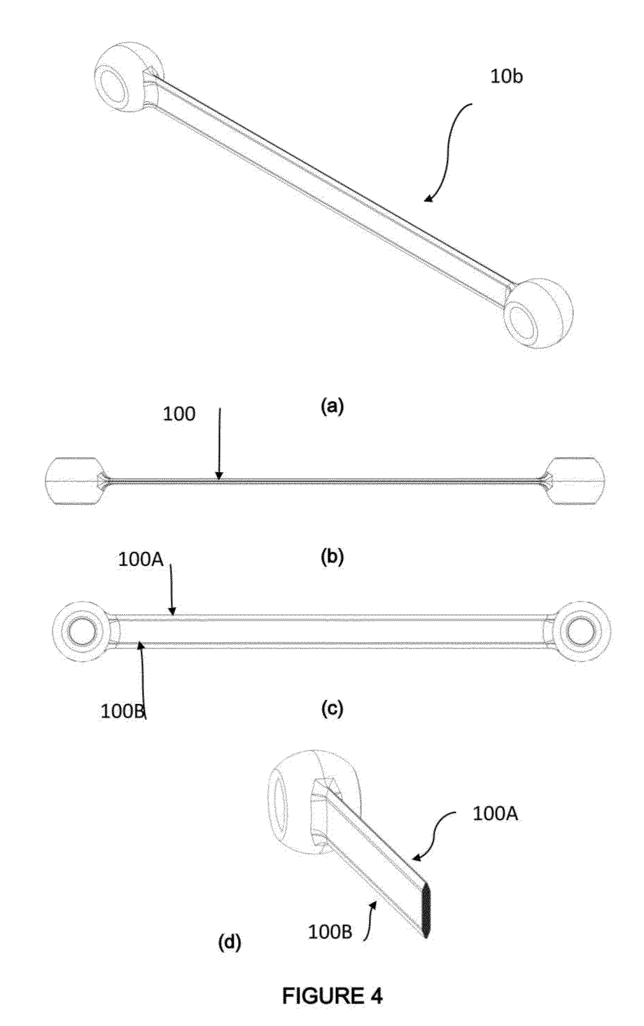

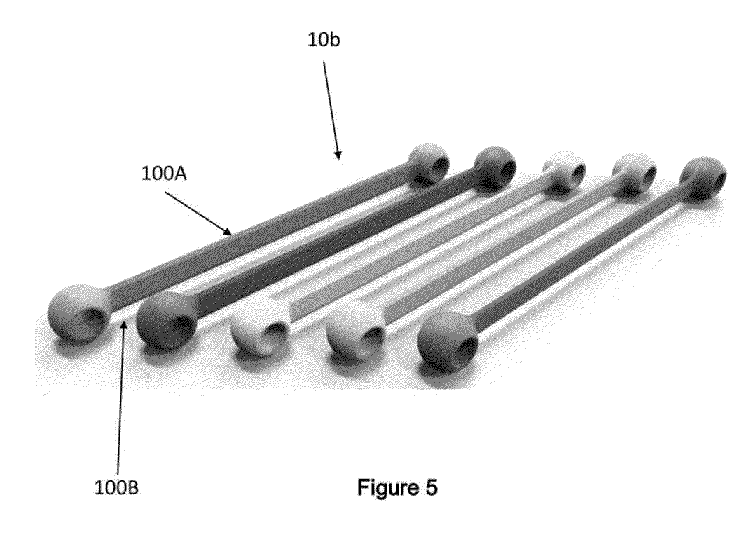

FIG. 4 illustrates a tongue cleaner 10b according to a second preferred embodiment of the present invention. The tongue cleaner 10b comprises a generally band shaped cross-section scraping member 100 which provides two oppositely disposed scraping edges being edges 100A and 1008. As shown in FIG. 5, the tongue cleaner 10 and 10b can be supplied in any number of attractive colours.

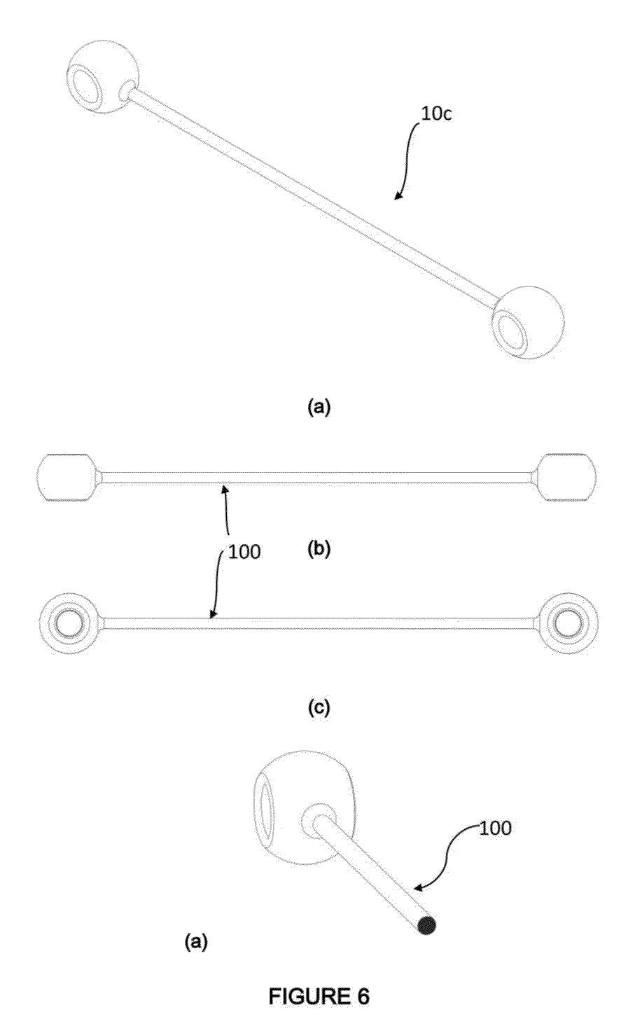

FIG. 6 illustrates a tongue cleaner 10c according to a third preferred embodiment of the present invention. The tongue cleaner 10c comprises a round shaped cross-section scraping member 100 which provides a smooth scraping edge around the scraping member 100. The scraping member can also have an oval cross-section.

1. A tongue cleaner comprising:
a scraping member having at least one scraping edge, the scraping member comprising a resilient flexible material allowing the scraping member to be bendable from a generally straight configuration into a curved or bent configuration.

2. A tongue cleaner as claimed in claim 1, wherein the scraping member defines two or more scraping edges.

3. A tongue cleaner as claimed in claim 2, wherein a first scraping edge has a different scraping characteristic to that of a second scraping edge.

4. A tongue cleaner as claimed in claim 2, wherein the scraping member bends into at least one of group consisting of a ‘C’ shape, ‘V’ shape and ‘U’ shape.

5. A tongue cleaner as claimed in claim 1, wherein the scraping member defines three scraping edges.

6. A tongue cleaner as claimed in claim 1, wherein the scraping member has a substantially planar portion.

7. A tongue cleaner as claimed in claim 1, wherein the scraping member has a generally T-shaped cross-section.

8. A tongue cleaner as claimed in claim 1, wherein the scraping member terminates at each end in a grip structure, the grip structure facilitating gripping and manipulation of the tongue cleaner.

9. A tongue cleaner as claimed in claim 8, wherein said grip structure defines an aperture passing therethrough, the aperture receiving a supporting element therein, said scraping member hanging on said supporting element.

10. A tongue cleaner as claimed in claim 8, wherein each said grip structure has a shape selected from at least one of a group consisting of: spherical, hemispherical, cubed, polygon, circular, heart shape, a knob handle, a diamond handle, a square handle, a rectangle handle and an oval handle.

11. A tongue cleaner as claimed in claim 1, wherein the scraping member is comprised of at least one of a group consisting of: a plastic, silicone, rubber, metal, wood and any combination thereof.

12. A tongue cleaner as claimed in claim 1, wherein a length of the scraping member is between 100 mm and 200 mm.

13. A tongue cleaner as claimed in claim 8, wherein a width of each grip structure is between 10 mm and 20 mm.

14. A tongue cleaner as claimed in claim 13, wherein a diameter of each grip structure is between 15 mm and 20 mm.

15. A tongue cleaner as claimed in claim 3, wherein the different scraping characteristic comprises at least one of a group consisting of: a different angle, shape, texture, hardness and contour.

16. A tongue cleaner as claimed in claim 1, wherein a material of the scraping member is treated with an anti-bacterial agent.

17. A tongue cleaner as claimed in claim 1, wherein the scraping member has a generally band-shaped cross-section.

18. A tongue cleaner as claimed in claim 1, wherein the scraping member has a generally round-shaped or oval cross-section.
